(19) United States
(12) Patent Application Publication
(10) Pub. No.: US 2022/0029647 A1
(43) Pub. Date: Jan. 27, 2022

(54) RECEPTION APPARATUS AND RECEPTION METHOD

(71) Applicant: OLYMPUS CORPORATION, Tokyo (JP)

(72) Inventor: Naoya HIGAKI, Tokyo (JP)

(73) Assignee: OLYMPUS CORPORATION, Tokyo (JP)

(21) Appl. No.: 17/493,104

(22) Filed: Oct. 4, 2021

Related U.S. Application Data
(63) Continuation of application No. PCT/JP2019/014966, filed on Apr. 4, 2019.

(51) Int. Cl. H04B 1/00; H01Q 1/27

(52) U.S. Cl. CPC H04B 1/0064; H01Q 1/273; H04B 1/0078

(57) ABSTRACT

A reception system includes a reception circuit, an antenna element, a cable, a switching circuit, and a control unit. The switching circuit can perform switching between a first connection state in which the reception circuit, the antenna element, and the cable are electrically connected to each other and a second connection state in which the reception circuit and the cable are electrically connected to each other but the antenna element is not electrically connected to the cable. The control unit controls the switching circuit such that the first connection state is obtained in a first time period and the second connection state is obtained in a second time period.

### CROSS REFERENCE TO RELATED APPLICATION

This application is a continuation application of PCT/JP2019/014966 filed on Apr. 4, 2019, the entire contents of which are incorporated herein by this reference.

### BACKGROUND OF THE INVENTION

### 1. Field of the Invention

The present invention relates to a reception apparatus that receives a radio signal transmitted from a mobile body such as a capsule endoscope, and a reception method of receiving the radio signal transmitted from the mobile body such as the capsule endoscope.

### 2. Description of the Related Art

In general, in a medical field, an endoscope is used for the purpose of observation of an inside of a living body and the like. In recent years, a capsule endoscope that is introduced into a body when an examinee swallows the capsule endoscope has been proposed as one type of an endoscope. The capsule endoscope generates image data by picking up an image of an object in a body cavity by moving in the body in accordance with peristaltic movement, generates a radio signal by encoding the image data in accordance with predetermined rules, and transmits the radio signal to an external reception system by wireless communication.

The reception system includes a plurality of antennas, and is configured to be able to detect a reception strength of a radio signal of each antenna. The reception strength is used to detect a position of the capsule endoscope and to select an antenna that receives the image data. For example, Japanese Patent Application Laid-Open Publication No. 2009-153617 discloses a reception apparatus that calculates position data of a capsule endoscope in a subject on the basis of a reception electric field strength acquired for each antenna. Japanese Patent Application Laid-Open Publication No. 2007-88860 discloses a reception apparatus that selects an antenna having the highest reception electric field strength as an antenna that receives a video signal transmitted from a capsule endoscope.

The antenna is configured by an antenna element serving as reception means for receiving a radio signal, and a cable electrically connected to the antenna element. The reception strength of the radio signal is calculated on the basis of the reception strength of the radio signal received by the antenna element.

### SUMMARY OF THE INVENTION

A reception apparatus of one aspect of the present invention is a reception apparatus including: a processor configured to perform predetermined processing on an electrical signal corresponding to a radio signal configured by a preamble portion and a data portion following the preamble portion, the data portion including image data picked up by a mobile body; and a switching circuit configured to be capable of performing switching between a first connection state in which an antenna element capable of receiving the radio signal receives the radio signal, and a second connection state in which only a cable positioned between the processor and the antenna element and configured to transmit the electrical signal receives the radio signal transmitted by the mobile body. In the reception apparatus, the processor is configured to: switch the switching circuit such that the first connection state is obtained in a first time period out of a time period in which the preamble portion is received and the second connection state is obtained in a second time period out of the time period in which the preamble portion is received, the second time period being different from the first time period; and switch the switching circuit to either the first connection state or the second connection state based on the electrical signal corresponding to the radio signal received in each of the first time period and the second time period in a time period in which the data portion is received.

A reception method of one aspect of the present invention is a reception method of receiving a radio signal configured by a preamble portion and a data portion following the preamble portion, the data portion including image data picked up by a mobile body, the radio signal being transmitted by the mobile body, the reception method including: switching the switching circuit to a first connection state in which an antenna element capable of receiving the radio signal receives the radio signal in a first time period out of a time period in which the preamble portion is received; switching the switching circuit to a second connection state in which only a cable positioned between a processor configured to perform predetermined processing on an electrical signal corresponding to the radio signal and the antenna element receives the radio signal in a second time period out of the time period in which the preamble portion is received, the second time period being different from the first time period, the cable being configured to transmit the electrical signal; and switching the switching circuit to either the first connection state or the second connection state based on the electrical signal corresponding to the radio signal received in each of the first time period and the second time period in a time period in which the data portion is received.

A reception apparatus of another aspect of the present invention is a reception apparatus configured to receive a radio signal configured by a preamble portion and a data portion following the preamble portion, the data portion including image data picked up by a mobile body, the radio signal being transmitted by the mobile body, the reception apparatus including: a processor configured to perform predetermined processing on an electrical signal corresponding to the radio signal; a reception antenna configured by an antenna element capable of receiving the radio signal, and a cable positioned between the processor and the antenna element and configured to transmit the electrical signal; and a switching circuit capable of performing switching between a first connection state in which the antenna element receives the radio signal and a second connection state in which only the cable receives the radio signal. In the reception apparatus, the processor: switches the switching circuit such that the first connection state is obtained in a first time period out of a time period in which the preamble portion is received and the second connection state is obtained in a second time period out of the time period in which the preamble portion is received, the second time period being different from the first time period; and switches the switching circuit to either the first connection state or the second connection state based on the electrical signal corresponding to the radio signal received in each of the first time period and the second time period in a time period in which the data portion is received.

### BRIEF DESCRIPTION OF THE DRAWINGS

FIG. 1 is an explanatory diagram illustrating a configuration of a capsule endoscope system including a reception system according to a first embodiment of the present invention;

FIG. 2 is an explanatory diagram illustrating an aspect of a usage of the capsule endoscope system in the first embodiment of the present invention;

FIG. 3 is a functional block diagram illustrating a configuration of a reception antenna of a reception system according to the first embodiment of the present invention;

FIG. 4 is a functional block diagram illustrating one example of a configuration of a first connection circuit in the first embodiment of the present invention;

FIG. 5 is a functional block diagram illustrating one example of a configuration of a second connection circuit in the first embodiment of the present invention;

FIG. 6 is a functional block diagram illustrating a second connection state in the first embodiment of the present invention;

FIG. 7 is an explanatory diagram schematically illustrating a configuration of a radio signal in the first embodiment of the present invention;

FIG. 8 is a flowchart illustrating a series of processing for receiving a data portion in the first embodiment of the present invention;

FIG. 9 is a functional block diagram illustrating a configuration of a reception system according to a second embodiment of the present invention;

FIG. 10 is a functional block diagram illustrating a configuration of a reception antenna of a reception system according to a third embodiment of the present invention;

FIG. 11 is a functional block diagram illustrating a configuration of a modification of the reception antenna of the reception system according to the third embodiment of the present invention; and

FIG. 12 is a functional block diagram illustrating a configuration of a reception antenna of a reception system according to a fourth embodiment of the present invention.

### DETAILED DESCRIPTION OF THE PREFERRED EMBODIMENTS

Embodiments of the present invention are described below with reference to the drawings.

### First Embodiment

### (Configuration of Endoscope System)

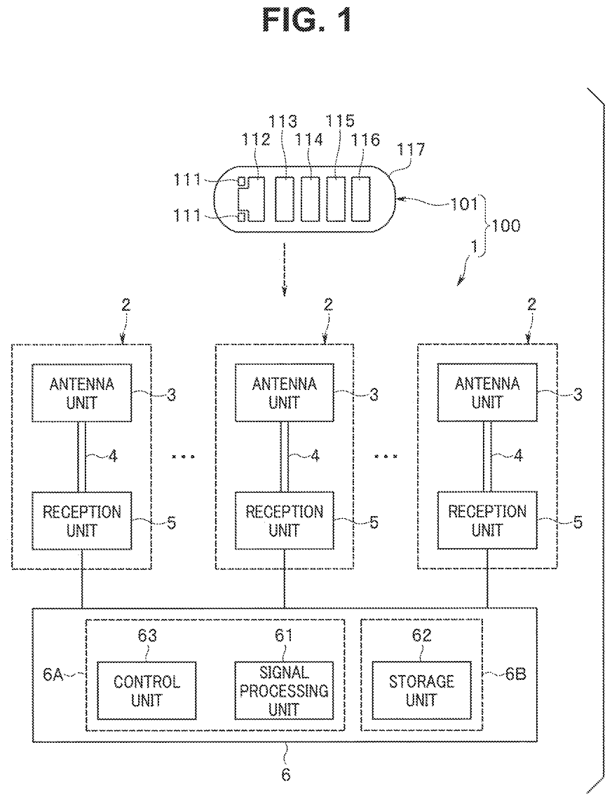

First, a configuration of a capsule endoscope system including a reception system according to a first embodiment of the present invention is described. FIG. 1 is an explanatory diagram illustrating the configuration of the capsule endoscope system in the present embodiment. As illustrated in FIG. 1, a capsule endoscope system 100 includes a capsule endoscope 101 serving as a mobile body and a reception system 1 according to the present embodiment.

The capsule endoscope 101 has dimensions and a shape with which the capsule endoscope 101 can be disposed in a body cavity of a subject, and is introduced into a lumen of a digestive system when the capsule endoscope 101 is swallowed by the subject. The capsule endoscope 101 is configured to be able to pick up an image of an inside of the subject, generate image data, and transmit the image data with the use of a radio signal. In the present embodiment, the capsule endoscope 101 includes illumination units 111, an image pickup unit 112, a wireless communication unit 113, a storage unit 114, a power supply unit 115, a control unit 116, and a housing 117. The housing 117 has an elongated capsule-like shape. Components of the capsule endoscope 101 besides the housing 117 are sealed in the housing 117.

Each of the illumination units 111 includes a light emitting element such as an LED, and generates illumination light for illuminating an object in the subject. The image pickup unit 112 includes an image pickup device such as a CCD, picks up an image of the object illuminated by the illumination units 111, and generates image data.

The wireless communication unit 113 includes a code modulation unit and an antenna, and transmits a radio signal encoded in accordance with predetermined rules. In FIG. 1, a dashed arrow indicates the radio signal. The radio signal includes information on the image data generated by the image pickup unit 112.

The wireless communication unit 113 may not only have a function of transmitting a radio signal but also may have a function of receiving a radio signal transmitted by the reception system 1. In the case, in the wireless communication unit 113, a demodulation unit that restores information from the received radio signal is provided.

The storage unit 114 is formed by a memory such as a RAM, and is configured to be able to store the image data generated by the image pickup unit 112 in the storage unit 114. The power supply unit 115 is formed by a battery, for example, and is configured to be able to supply drive power to each unit of the capsule endoscope 101. The control unit 116 controls an operation of each unit of the capsule endoscope 101.

The reception system 1 is physically separated from the capsule endoscope 101. In the present embodiment, the reception system 1 includes a plurality of reception antennas 2 and a reception apparatus 6. The reception apparatus 6 may be configured as a stationary apparatus such as a workstation or may be configured as a portable terminal apparatus driven by a battery.

Each of the plurality of reception antennas 2 is configured to be able to receive the radio signal transmitted by the wireless communication unit 113 of the capsule endoscope 101, and output an electrical signal (hereinafter referred to as a reception signal) corresponding to the received radio signal to the reception apparatus 6. Each configuration of the plurality of reception antennas 2 is the same. In an example illustrated in FIG. 1, each of the plurality of reception antennas 2 includes an antenna unit 3, a reception unit 5, and a cable 4 that connects components of the antenna unit 3 and components of the reception unit 5 to each other. In the present embodiment, the cable 4 is configured to transmit a reception signal by a single-ended method. As the cable 4, a coaxial cable is used, for example. The reception unit 5 is connected to the reception apparatus 6 by a signal line.

The reception apparatus 6 includes a signal processing unit 61, a storage unit 62, and a control unit 63. The signal processing unit 61 performs predetermined processing on the reception signal. In the predetermined processing, processing of acquiring the image data included in the reception signal is included. The storage unit 62 is configured to be able to store the acquired image data. The control unit 63 controls the signal processing unit 61 and the storage unit 62 of the reception apparatus 6, and controls a control unit included in each of the plurality of reception antennas 2 described below.

The reception system 1 may further include a display unit (not shown) configured by a display apparatus such as an LCD. The display unit is connected to the reception apparatus 6, and displays an image corresponding to the image data acquired by the signal processing unit 61.

Hardware configuring the signal processing unit 61, the storage unit 62, and the control unit 63 is described. In the reception apparatus 6, a processor 6A, a storage apparatus 6B, and an input-output unit are mounted. The processor 6A is configured by an FPGA (field programmable gate array), for example. At least one part of the signal processing unit 61 and the control unit 63 may be configured as a circuit block in the FPGA. The storage apparatus 6B includes a memory such as a RAM, and a rewritable storage such as a flash memory or a magnetic disk apparatus. The storage unit 62 may be configured by a memory or may be configured by a storage. The input-output unit is used for transmission and reception of signals between the reception apparatus 6 and an outside.

The processor 6A may be configured by a central processing unit (hereinafter referred to as a CPU). In the case, at least one part of the signal processing unit 61 and the control unit 63 may be realized by reading out a program from the storage apparatus 6B and executing the program by the CPU.

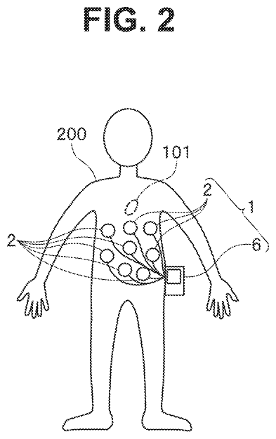

Next, an aspect of a usage of the capsule endoscope system 100 is described by taking a case where the reception apparatus 6 is configured as a portable terminal apparatus as an example. In the case, the reception apparatus 6 is configured such that a battery (not shown) is installed. FIG. 2 is a schematic diagram illustrating an aspect of the usage of the capsule endoscope system 100. At the time of the usage of the capsule endoscope system 100, the capsule endoscope 101 is disposed in the body cavity of a subject 200, the reception apparatus 6 is installed on an outside of a body of the subject 200, and the plurality of reception antennas 2 are disposed on a body surface portion of the subject 200.

### (Configuration of Reception Antenna)

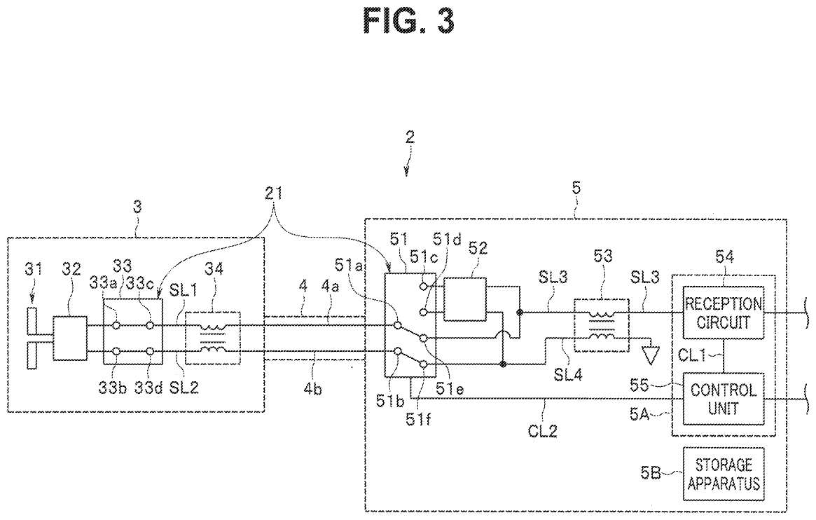

Next, a configuration of the reception antenna 2 is described by focusing on one reception antenna 2. FIG. 3 is a functional block diagram illustrating a configuration of the reception antenna 2. As described above, the reception antenna 2 includes the antenna unit 3, the cable 4, and the reception unit 5. The reception antenna 2 further includes a switching circuit 21. The switching circuit 21 includes a first switching circuit unit 33 and a second switching circuit unit 51.

The antenna unit 3 includes an antenna element 31, a first connection circuit 32, a first switching circuit unit 33, and a first separation circuit 34. In terms of circuit configuration, the first connection circuit 32, the first switching circuit unit 33, and the first separation circuit 34 are positioned between the antenna element 31 and the cable 4 and are arranged in the stated order from the antenna element 31 side.

The antenna element 31 is reception means for receiving a radio signal and converting the received radio signal into a reception signal. As the antenna element 31, various shapes of antenna elements are used. In the present embodiment, a balanced dipole antenna is used as the antenna element 31. The balanced dipole antenna converts a received radio signal into a differential signal. As the antenna element 31, instead of the dipole antenna illustrated in FIG. 3, an antenna having radiation properties equivalent to radiation properties of the dipole antenna may be used. More specifically, deformed dipole antennas such as a folded dipole antenna and a meander line antenna may be used as the antenna element 31.

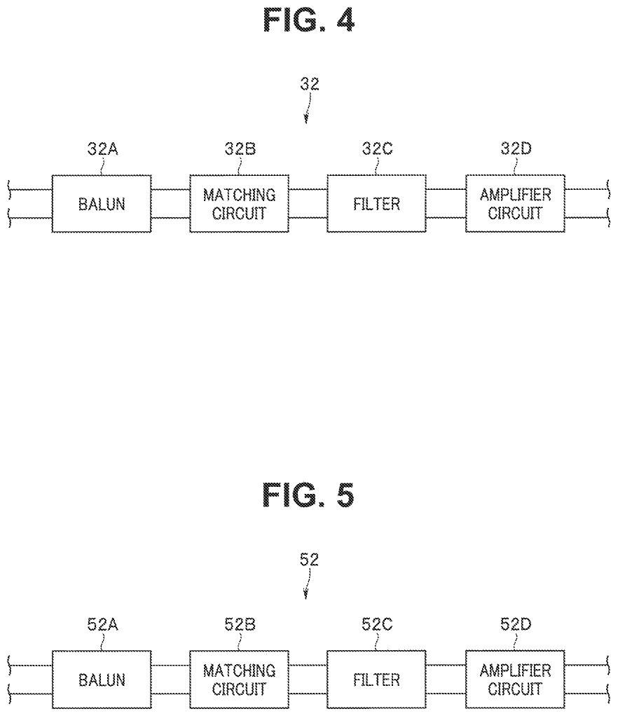

The first connection circuit 32 is a circuit for connecting the antenna element 31 to an electrical circuit of the reception antenna 2. FIG. 4 illustrates one example of a configuration of the first connection circuit 32. As illustrated in FIG. 4, the first connection circuit 32 may include a balun 32A, a matching circuit 32B, a filter 32C, and an amplifier circuit 32D. In terms of circuit configuration, the balun 32A, the matching circuit 32B, the filter 32C, and the amplifier circuit 32D are arranged in the stated order from the antenna element 31 side. The balun 32A is electrically connected to the antenna element 31. The amplifier circuit 32D is electrically connected to the first switching circuit unit 33.

The balun 32A converts the differential signal converted from the radio signal by the antenna element 31 to a single-ended signal. In the present embodiment, the antenna element 31 is a dipole antenna that converts a received radio signal to a differential signal, and the cable 4 is configured to transmit the reception signal by the single-ended method. Therefore, in the present embodiment, the balun 32A is an essential component of the first connection circuit 32.

The matching circuit 32B performs impedance matching between the antenna element 31 and the electrical circuit of the reception antenna 2. The filter 32C is a bandpass filter, for example, and removes signals other than the radio signal such as interfering waves. The amplifier circuit 32D amplifies an energy of the reception signal, and adjusts a noise figure that is a ratio of an S/N ratio of an inputted signal to an S/N ratio of an outputted signal to a predetermined value.

The first connection circuit 32 does not necessarily need to include all of the components illustrated in FIG. 4, and components necessary for the first connection circuit 32 may be selected from the components illustrated in FIG. 4 on the basis of the type of the antenna element 31, a circuit configuration of the reception antenna 2 besides the first connection circuit 32, and the like.

The first switching circuit unit 33 is a switch circuit having two contacts 33a and 33b on an input side, and two contacts 33c and 33d on an output side. The first switching circuit unit 33 is configured to be able to control opening and closing between the contacts 33a and 33c and opening and closing between contacts 33b and 33d. In FIG. 3, a state is illustrated in which the contact 33a and the contact 33c are connected to each other and the contact 33b and the contact 33d are connected to each other.

In the present embodiment, in particular, the reception signal (single-ended signal) is inputted to the contact 33a. A signal line electrically connected to the contacts 33a and 33c in the antenna unit 3 is referred to as a first signal line SL1, and a signal line electrically connected to the contacts 33b and 33d in the antenna unit 3 is referred to as a second signal line SL2 below. An opening and closing operation of the first switching circuit unit 33 is controlled by a reference potential of the first signal line SL1, that is, a reference potential of the single-ended signal, for example.

The first separation circuit 34 is a circuit for removing a noise signal transmitted to the antenna element 31 from the cable 4. For example, as illustrated in FIG. 3, the first separation circuit 34 may be a circuit in which a common mode choke coil is inserted in the first and second signal lines SL1 and SL2. The common mode choke coil may be a ferrite core using ferrite for a core. Alternatively, the first separation circuit 34 may be a ferrite cable (also referred to as an isolation cable) in which the first and second signal lines SL1 and SL2 are covered with ferrite.

The cable 4 includes a first conductor 4a electrically connected to the first signal line SL1, and a second conductor 4b electrically connected to the second signal line SL2. The reception signal (single-ended signal) is transmitted by the first conductor 4a.

In the present embodiment, the cable 4 can function as reception means for receiving a radio signal and converting the received radio signal into a reception signal. In the present embodiment, in particular, the cable 4 can function as a monopole antenna. When the cable 4 functions as the reception means, the received radio signal is converted into a single-ended signal by a balun of a second connection circuit described below, and is transmitted to the reception unit 5.

When the coaxial cable is used as the cable 4, the first conductor 4a is an internal conductor, and the second conductor 4b is an external conductor. In the case, the radio signal is received by the second conductor 4b, and an electrical signal in the same phase corresponding to the received radio signal is generated in the first conductor 4a and the second conductor 4b.

The reception unit 5 includes the second switching circuit unit 51, a second connection circuit 52, a second separation circuit 53, a reception circuit 54, and a control unit 55.

The second switching circuit unit 51 is a switch circuit having two contacts 51a and 51b on an input side, and four contacts 51c, 51d, 51e, and 51f on an output side. The second switching circuit unit 51 is configured to be able to select a connection destination of the contact 51a from the contacts 51c and 51e, and a connection destination of the contact 51b from the contacts 51d and 51f. FIG. 3 illustrates a state in which the contact 51a and the contact 51e are connected to each other and the contact 51b and the contact 51f are connected to each other.

The first conductor 4a of the cable 4 is electrically connected to the contact 51a. The second conductor 4b of the cable 4 is electrically connected to the contact 51b. Hereinafter, a signal line electrically connected to the contact 51e in the reception unit 5 is referred to as a third signal line SL3, and a signal line electrically connected to the contact 51f in the reception unit 5 is referred to as a fourth signal line SL4. In the state illustrated in FIG. 3, the third signal line SL3 is electrically connected to the first conductor 4a via the second switching circuit unit 51. The fourth signal line SL4 is electrically connected to the second conductor 4b via the second switching circuit unit 51.

In terms of circuit configuration, the second separation circuit 53 is positioned between each of the contacts 51c, 51d, 51e, and 51f of the second switching circuit unit 51 and the reception circuit 54. In terms of circuit configuration, the second connection circuit 52 is positioned between each of the contacts 51c and 51d of the second switching circuit unit 51 and the second separation circuit 53.

The second connection circuit 52 is a circuit for connecting the cable 4 serving as the reception means to the electrical circuit of the reception antenna 2. FIG. 5 illustrates one example of a configuration of the second connection circuit 52. As illustrated in FIG. 5, the second connection circuit 52 may include a balun 52A, a matching circuit 52B, a filter 52C, and an amplifier circuit 52D. In terms of circuit configuration, the balun 52A, the matching circuit 52B, the filter 52C, and the amplifier circuit 52D are arranged in the stated order from the second switching circuit unit 51 side. The balun 52A is electrically connected to the contacts 51c and 51d of the second switching circuit unit 51. The amplifier circuit 52D is electrically connected to the third and fourth signal lines SL3 and SL4.

The balun 52A converts a radio signal received by the cable 4 into a single-ended signal. The matching circuit 52B performs impedance matching between the cable 4 and the electrical circuit of the reception antenna 2 when the cable 4 functions as the reception means. The filter 52C is a bandpass filter, for example, and removes signals other than the radio signal such as interfering waves. The amplifier circuit 52D amplifies an energy of the reception signal, and adjusts a noise figure to a predetermined value.

The second connection circuit 52 does not necessarily need to include all of the components illustrated in FIG. 5, and components necessary for the second connection circuit 52 may be selected from the components illustrated in FIG. 5 on the basis of a structure of the cable 4, a circuit configuration of the reception antenna 2 besides the second connection circuit 52, and the like.

The second separation circuit 53 is a circuit for removing a noise signal transmitted from the reception circuit 54 to the cable 4. For example, as illustrated in FIG. 3, the second separation circuit 53 may be a circuit in which a winding balun is inserted in the third and fourth signal lines SL3 and SL4. The winding balun may be a ferrite core using ferrite for a core. Alternatively, the second separation circuit 53 may be a ferrite cable in which the third and fourth signal lines SL3 and SL4 are covered with ferrite. The fourth signal line SL4 is connected to a ground via the second separation circuit 53.

The reception circuit 54 is electrically connected to the third signal line SL3. The third signal line SL3 transmits a reception signal (single-ended signal). The reception circuit 54 is a circuit that performs predetermined processing on the reception signal and outputs the reception signal to the signal processing unit 61 (see FIG. 1) of the reception apparatus 6. In the present embodiment, the reception circuit 54 performs processing of calculating an index relating to a reception performance of the radio signal as the predetermined processing. Contents of the processing are described below.

The control unit 55 controls the first switching circuit unit 33 and the second switching circuit unit 51 of the switching circuit 21. In the present embodiment, a first control line CL1 connecting the reception circuit 54 and the control unit 55 to each other, and a second control line CL2 connecting the second switching circuit unit 51 and the control unit 55 to each other are provided. The control unit 55 controls an operation of the first switching circuit unit 33 by controlling the reception circuit 54 so as to cause a reference potential of the third signal line SL3, that is, a reference potential of the single-ended signal to be changed by the first control line CL1. The control unit 55 also controls an operation of the second switching circuit unit 51 by the second control line CL2.

The reception circuit 54 and the control unit 55 are connected to the control unit 63 (see FIG. 1) of the reception apparatus 6. At least the control unit 55 out of the reception circuit 54 and the control unit 55 is controlled by the control unit 63.

Hardware configuring the reception circuit 54 and the control unit 55 is described. In the reception unit 5, a processor 5A, a storage apparatus 5B, and an input-output unit are mounted. The processor is configured by an FPGA, for example. At least one part of the reception circuit 54 and the control unit 55 may be configured as a circuit block in the FPGA. The storage apparatus 5B includes a memory such as a RAM. The input-output unit is used for performing transmission and reception of signals between the reception unit 5 and the reception apparatus 6.

The processor 6A may be configured by an ASIC (application specific integrated circuit). In the case, at least one part of the reception circuit 54 and the control unit 55 is configured by the ASIC.

As described above, the reception system 1 according to the present embodiment includes the antenna element 31 capable of receiving the radio signal, the reception circuit 54, the cable 4 positioned between the reception circuit 54 and the antenna element 31 in terms of circuit configuration, the switching circuit 21, and the control unit 55. The reception system 1 further includes the plurality of reception antennas 2. The plurality of reception antennas 2 each include the antenna element 31, the cable 4, the switching circuit 21, and the control unit 55. The switching circuit 21 includes the first switching circuit unit 33 positioned between the antenna element 31 and the cable 4 in terms of circuit configuration, and the second switching circuit unit 51 positioned between the reception circuit 54 and the cable 4 in terms of circuit configuration. The reception system 1 further includes the first separation circuit 34 positioned between the antenna element 31 and the cable 4 in terms of circuit configuration, the second separation circuit 53 positioned between the reception circuit 54 and the cable 4 in terms of circuit configuration, and the matching circuit 52B positioned between the reception circuit 54 and the cable 4 in terms of circuit configuration.

(Operations of Switching Circuit, Reception Circuit, and Control unit) Next, operations of the switching circuit 21, the reception circuit 54 and the control unit 55 are described. First, connection states realized by the switching circuit 21 are described. The switching circuit 21 can perform switching between a first connection state in which the reception circuit 54, the antenna element 31, and the cable 4 are electrically connected to each other and a second connection state in which the reception circuit 54 and the cable 4 are electrically connected to each other but the antenna element 31 is not electrically connected to the cable 4.

FIG. 3 illustrates the first connection state. In the first connection state, in the first switching circuit unit 33 of the switching circuit 21, the contact 33a and the contact 33c are connected to each other, and the contact 33b and the contact 33d are connected to each other. As a result, the antenna element 31 and the cable 4 are placed in a state of being electrically connected to each other via the first connection circuit 32, the first switching circuit unit 33, and the first separation circuit 34.

In the first connection state, in the second switching circuit unit 51 of the switching circuit 21, the contact 51a and the contact 51e are connected to each other, and the contact 51b and the contact 51f are connected to each other. As a result, the reception circuit 54 and the cable 4 are placed in a state of being electrically connected to each other via the second switching circuit unit 51 and the second separation circuit 53.

As described above, by electrically connecting the antenna element 31 and the cable 4 to each other and electrically connecting the reception circuit 54 and the cable 4 to each other, a first connection state in which the reception circuit 54, the antenna element 31, and the cable 4 are electrically connected to each other is obtained. In the first connection state, the radio signal is substantially received by the antenna element 31. In other words, in the first connection state, only the radio signal received by the antenna element 31 becomes valid. The first connection state is also referred to as an antenna element reception state below.

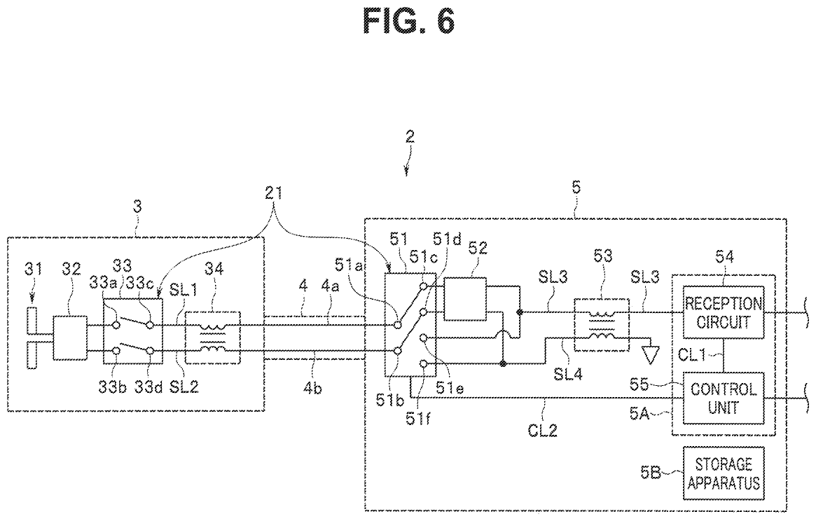

FIG. 6 illustrates the second connection state. In the second connection state, in the first switching circuit unit 33, a place between the contact 33a and the contact 33c is opened, and a place between the contact 33b and the contact 33d is opened. As a result, the antenna element 31 and the cable 4 are placed in a state of not being electrically connected to each other.

In the second connection state, in the second switching circuit unit 51, the contact 51a and the contact 51c are connected to each other, and the contact 51b and the contact 51d are connected to each other. As a result, the reception circuit 54 and the cable 4 are placed in a state of being electrically connected to each other via the second switching circuit unit 51, the second connection circuit 52, and the second separation circuit 53.

In the second connection state, by the first switching circuit unit 33, the antenna element 31 is placed in a state of being electrically disconnected from the electrical circuit of the reception antenna 2 including the reception circuit 54 and the cable 4. In the state, in place of the antenna element 31, the cable 4 functions as the reception means for receiving a radio signal and converting the received radio signal into a reception signal. In other words, in the second connection state, the radio signal is substantially received by the cable 4. In other words, in the second connection state, only the radio signal received by the cable 4 becomes valid. The second connection state is also referred to as a cable reception state below.

As illustrated in FIG. 5, the second connection circuit 52 includes the matching circuit 52B. By the second switching circuit unit 51, the reception circuit 54 and the cable 4 are electrically connected to each other without passing through the matching circuit 52B in the antenna element reception state, but are electrically connected to each other via the matching circuit 52B in the cable reception state. Therefore, in the antenna element reception state, that is, when the cable 4 does not function as the reception means, the matching circuit 52B does not perform impedance matching. In the cable reception state, that is, when the cable 4 functions as the reception means, the matching circuit 52B performs impedance matching.

Description of the matching circuit 52B is also applicable to other components of the second connection circuit 52.

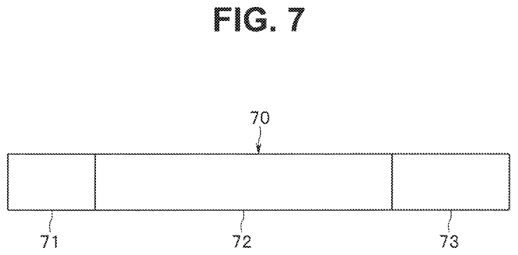

Next, the operations of the reception circuit 54 and the control unit 55 are described. The control unit 55 controls the switching circuit 21 such that the antenna element reception state is obtained in a first time period in which the radio signal is received, and the cable reception state is obtained in a second time period different from the first time period in which the radio signal is received. One example of a configuration of the radio signal is described with reference to FIG. 7. FIG. 7 schematically illustrates the configuration of the radio signal.

In the example illustrated in FIG. 7, a radio signal 70 includes a preamble portion 71, a data portion 72 following the preamble portion 71, and a position detection portion 73. In the example, the preamble portion 71 is positioned in a head portion of the radio signal 70, and the position detection portion 73 is positioned in a tail portion of the radio signal 70. The position of the position detection portion 73 is not limited to the example illustrated in FIG. 7.

The preamble portion 71 includes identification data that changes with a predetermined pattern. The data portion 72 includes image data picked up by the image pickup unit 112 (see FIG. 1). The position detection portion 73 is a portion for detecting a position of the capsule endoscope 101 (see FIG. 1), and may include data that changes with a predetermined pattern or may be an unmodulated signal, for example.

Both of the first and second time periods are included in a time period in which the preamble portion 71 is received. The reception circuit 54 calculates an index (hereinafter referred to as a reception index) relating to a reception performance of the radio signal on the basis of the reception signal in each of the first and second time periods. In more detail, the reception circuit 54 calculates a reception index on the basis of a reception signal corresponding to a radio signal received by the antenna element 31 in the first time period, and calculates a reception index on the basis of a reception signal corresponding to a radio signal received by the cable 4 in the second time period.

The control unit 55 selects either the first connection state (antenna element reception state) or the second connection state (cable reception state) on the basis of the reception indices, and controls the switching circuit 21 such that the selected connection state is obtained in a time period in which the data portion 72 is received. In the present embodiment, the reception index is an index having correspondence with an ease of reception of the radio signal by the reception antenna 2. The control unit 55 selects a connection state in which the radio signal is received more easily on the basis of the reception index.

The reception index may be a reception strength of the radio signal. The reception strength of the radio signal is expressed by a magnitude of electric power of the reception signal, for example. In the case, the control unit 55 selects a connection state corresponding to a time period in which a radio signal having a larger reception strength is received out of the first time period and the second time period.

Alternatively, the control unit 55 may read reference data serving as a reference for the identification data of the preamble portion 71 from the storage apparatus 5B or the storage apparatus 6B and hold the reference data. In the case, the reception index may be a rate of concordance between the reference data and the identification data. In the case, the control unit 55 selects a connection state corresponding to a time period in which a radio signal having a higher rate of concordance is received out of the first time period and the second time period.

In the present embodiment, in particular, the plurality of reception antennas 2 are provided, and the control unit 55 is provided for each of the plurality of reception antennas 2. The control unit 55 of each of the plurality of reception antennas 2 is controlled by the control unit 63 of the reception apparatus 6. The control unit 63 selects the reception antenna 2 that receives the data portion on the basis of the reception index. As described above, in the present embodiment, the reception index is an index having a correspondence with an ease of reception of the radio signal by the reception antenna 2. The control unit 63 selects the reception antenna 2 that receives the radio signal more easily on the basis of the reception index. The control unit 63 indirectly controls the switching circuit 21 by controlling the control unit 55 of the selected reception antenna 2 such that the selected connection state is obtained in the selected reception antenna 2.

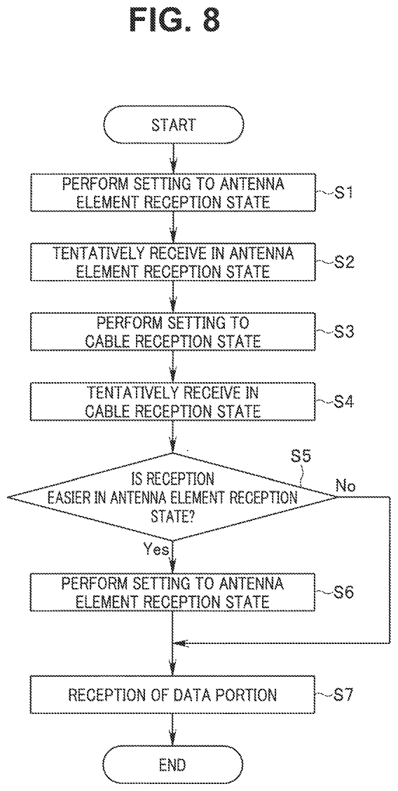

A series of processing for receiving the data portion 72 is described by focusing on one reception antenna 2. FIG. 8 is a flowchart illustrating the series of processing for receiving the data portion 72.

As illustrated in FIG. 8, in the series of processing, first, the reception antenna 2 is set to the antenna element reception state (Step S1). More specifically, the control unit 55 controls the switching circuit 21 such that the antenna element reception state is obtained in the first time period out of the time period in which the preamble portion 71 is received. Next, the radio signal is tentatively received in the antenna element reception state (Step S2). More specifically, the antenna element 31 receives the radio signal. The reception circuit 54 calculates the reception index on the basis of the reception signal corresponding to the radio signal.

Next, the reception antenna 2 is set to the cable reception state (Step S3). More specifically, the control unit 55 controls the switching circuit 21 such that the cable reception state is obtained in the second time period out of the time period in which the preamble portion 71 is received. Next, the radio signal is tentatively received in the cable reception state (Step S4). More specifically, the cable 4 receives the radio signal. The reception circuit 54 calculates the reception index on the basis of the reception signal corresponding to the radio signal.

Next, the control unit 55 determines whether reception is easier in the antenna element reception state (Step S5). When the reception index is the reception strength of the radio signal, the control unit 55 determines that reception is easier in the antenna element reception state when the reception strength of the radio signal received in the antenna element reception state is larger than the reception strength of the radio signal received in the cable reception state. When the reception index is the rate of concordance between the reference data and the identification data, the control unit 55 determines that reception is easier in the antenna element reception state when the rate of concordance of the radio signal received in the antenna element reception state is higher than the rate of concordance of the radio signal received in the cable reception state.

When it is determined that reception is easier in the antenna element reception state in Step S5 (YES), the reception antenna 2 is set to the antenna element reception state next (Step S6). When it is not determined that reception is easier in the antenna element reception state in Step S5 (NO), the reception antenna 2 stays in the cable reception state. Next, the reception antenna 2 receives the data portion 72 (Step S7), and the series of processing ends.

Next, the operations of the control units 55 and 63 in the time period in which the position detection portion 73 is received are described. In the time period in which the position detection portion 73 is received, the control unit 55 controls the switching circuit 21 such that the antenna element reception state is obtained. In the present embodiment, in particular, in the time period in which the position detection portion 73 is received, the control unit 63 indirectly controls the switching circuit 21 by controlling the control unit 55 of the reception antenna 2 such that the antenna element reception state is obtained in all of the plurality of reception antennas 2.

### (Effects)

Next, effects of the reception system 1 according to the present embodiment are described. In the present embodiment, the switching circuit 21 can perform switching between the antenna element reception state and the cable reception state by the control of the control unit 55. In general, the radio signal transmitted from the capsule endoscope 101 is received by the antenna element 31. However, there may be cases where the radio signal cannot be received by the antenna element 31 but can be received by the cable 4 depending on the position of the capsule endoscope 101. In the present embodiment, in cases as above, the radio signal can be received by the cable 4 by controlling the switching circuit 21 such that the cable reception state is obtained. As above, according to the present embodiment, by performing switching between the antenna element reception state and the cable reception state in accordance with the position of the capsule endoscope 101, a reception range of the radio signal can be expanded as compared to a case of a configuration in which the radio signal is only received by the antenna element 31.

In the present embodiment, the reception index in each of a case of the antenna element reception state and a case of the cable reception state is calculated by performing switching between the antenna element reception state and the cable reception state in the time period in which the preamble portion 71 (see FIG. 7) of the radio signal 70 is received. The control unit 55 selects the state in which the radio signal is easily received out of the antenna element reception state and the cable reception state on the basis of the calculated reception indices, and controls the switching circuit 21 such that the selected reception state (connection state) is obtained in the time period in which the data portion 72 (see FIG. 7) of the radio signal 70 is received. As a result, according to the present embodiment, the data portion 72 can be received in an optimal reception state (connection state).

In the present embodiment, the plurality of reception antennas 2 are provided, and the reception indices are calculated for each of the plurality of reception antennas 2 as above. The control unit 55 selects the reception antenna 2 that receives the data portion 72 on the basis of the reception indices. By the above, according to the present embodiment, the data portion 72 can also be received in the optimal reception state (connection state).

In the present embodiment, the control unit 55 controls the switching circuit 21 such that the antenna element reception state is obtained in the time period in which the position detection portion 73 (see FIG. 7) of the radio signal 70 is received. In the present embodiment, in particular, the switching circuit 21 is controlled such that the antenna element reception state is obtained in all of the plurality of reception antennas 2. As a result, according to the present embodiment, the reception strength of the radio signal can be prevented from changing. As a result, a detection accuracy of the capsule endoscope 101 can be improved.

In the present embodiment, the first separation circuit 34 for removing the noise signal transmitted to the antenna element 31 from the cable 4 is provided between the antenna element 31 and the cable 4. As a result, according to the present embodiment, the influence of the noise signal in the antenna element 31 can be reduced. As a result, the reception strength of the radio signal received by the antenna element 31 can be prevented from changing. By the above, according to the present embodiment, the detection accuracy of the capsule endoscope 101 can also be improved.

In the present embodiment, the second separation circuit 53 for removing the noise signal transmitted to the cable 4 from the reception circuit 54 is provided between the reception circuit 54 and the cable 4. As a result, according to the present embodiment, the influence of the noise signal in the cable reception state can be reduced.

From the above, according to the present embodiment, the improvement of the detection accuracy of the position of the capsule endoscope 101 and the expansion of the reception range of the radio signal transmitted by the capsule endoscope 101 can be compatible with each other.

In order to reduce the influence of the noise signal in the reception antenna 2, a case where the cable 4 is configured to transmit the reception signal by a differential method can also be conceived. However, in the case, a circuit for processing a differential signal is needed. In the present embodiment, the cable 4 is configured to transmit the reception signal by the single-ended method. As a result, according to the present embodiment, a circuit for processing a differential signal becomes unnecessary, and a circuit configuration of the reception antenna 2 can be simplified.

In the present embodiment, by configuring the cable 4 such that the cable 4 transmits the reception signal by the single-ended method, the control of the first switching circuit unit 33 of the switching circuit 21 disposed in the antenna unit 3 can be performed with the use of the signal line through which the reception signal (single-ended signal) is transmitted, and the control line for controlling the first switching circuit unit 33 can be omitted. As described above, the control of the first switching circuit unit 33 can be controlled by a reference potential of the single-ended signal, for example.

In the present embodiment, the matching circuit 32B that performs impedance matching between the antenna element 31 and the electrical circuit of the reception antenna 2 is provided between the antenna element 31 and the cable 4, and the matching circuit 52B that performs impedance matching between the cable 4 and the electrical circuit of the reception antenna 2 is provided between the reception circuit 54 and the cable 4. In the antenna element reception state, the reception circuit 54 and the cable 4 are electrically connected to each other without passing through the matching circuit 52B. In the case, the matching circuit 32B performs impedance matching, but the matching circuit 52B does not perform impedance matching. In the cable reception state, the matching circuit 32B is placed in a state of being disconnected from the electrical circuit of the reception antenna 2 including the reception circuit 54, and the reception circuit 54 and the cable 4 are electrically connected to each other via the matching circuit 52B. In the case, the matching circuit 32B does not perform impedance matching, but the matching circuit 52B performs impedance matching. As above, according to the present embodiment, optimal impedance matching can be performed in each of the antenna element reception state and the cable reception state, and electric power of the reception signal can be prevented from being consumed in the matching circuit 52B in the antenna element reception state.

### Second Embodiment

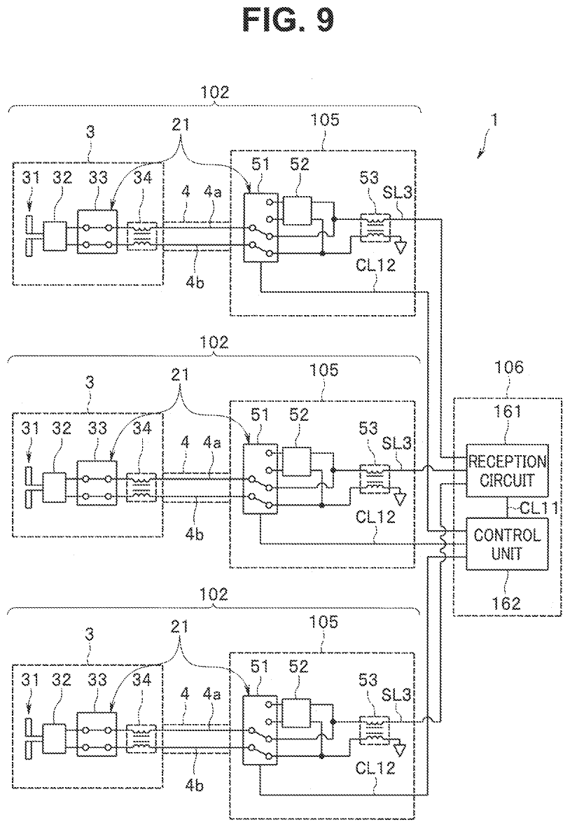

Next, with reference to FIG. 9, a second embodiment of the present invention is described. FIG. 9 is a functional block diagram illustrating a configuration of the reception system 1 according to the present embodiment. The configuration of the reception system 1 according to the present embodiment is different from the first embodiment in the following point. The reception system 1 according to the present embodiment includes a plurality of reception antennas 102 and a reception apparatus 106 instead of the plurality of reception antennas 2 and the reception apparatus 6 in the first embodiment. In FIG. 9, three reception antennas 102 are illustrated for the sake of convenience. However, the number of the plurality of reception antennas 102 is not limited to the example illustrated in FIG. 9.

A configuration of the plurality of reception antennas 102 is the same. Each of the plurality of reception antennas 102 includes the antenna unit 3, the reception unit 105, the cable 4 that connects the components of the antenna unit 3 and the components of the reception unit 105 to each other, and the switching circuit 21. The reception unit 105 is connected to the reception apparatus 106 by a signal line. Configurations of the antenna unit 3, the cable 4, and the switching circuit 21 are the same as the configurations of the antenna unit 3, the cable 4, and the switching circuit 21 in the first embodiment.

A configuration of the reception unit 105 is basically the same as the configuration of the reception unit 5 in the first embodiment except for a feature in which the reception circuit 54 and the control unit 55 are not provided. In other words, the reception unit 105 includes the second switching circuit unit 51, the second connection circuit 52, and the second separation circuit 53 as with the reception unit 5.

As with the first embodiment, the signal line that transmits the reception signal (single-ended signal) in the reception unit 105 is referred to as the third signal line SL3. The third signal line SL3 extends to the reception apparatus 106 from the reception antenna 102.

The reception apparatus 106 includes a reception circuit 161 and a control unit 162. The reception circuit 161 is electrically connected to the third signal lines SL3 that each extend from each of the plurality of reception antennas 102. A function of the reception circuit 161 is the same as a function of the reception circuit 54 in the first embodiment.

The control unit 162 controls the first switching circuit unit 33 and the second switching circuit unit 51 of the switching circuit 21 of each of the plurality of reception antennas 102. In the present embodiment, a first control line CL11 connecting the reception circuit 161 and the control unit 162 to each other, and a plurality of second control lines CL12 that each connect the second switching circuit unit 51 of each of the plurality of reception antennas 102 and the control unit 162 to each other are provided. The control unit 162 controls an operation of the first switching circuit unit 33 of each of the plurality of reception antennas 102 by controlling the reception circuit 161 so as to change a reference potential of the third signal line SL3, that is, a reference potential of the single-ended signal of each of the plurality of reception antennas 102 by the first control line CL11. The control unit 162 controls an operation of the second switching circuit unit 51 of each of the plurality of reception antennas 102 by the plurality of second control lines CL12.

Although not shown, the reception apparatus 106 further includes the signal processing unit 61 and the storage unit 62 described in the first embodiment. The signal processing unit 61 and the storage unit 62 in the present embodiment are controlled by the control unit 162. The reception circuit 161 outputs a reception signal to the signal processing unit 61.

Effects of other configurations in the present embodiment are similar to the effects of the first embodiment.

### Third Embodiment

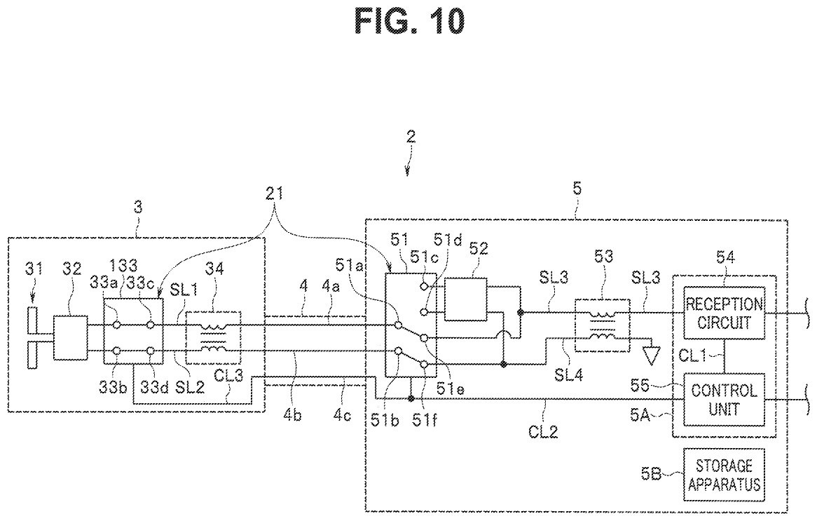

Next, a third embodiment of the present invention is described with reference to FIG. 10. FIG. 10 is a functional block diagram illustrating a configuration of the reception antenna 2 in the present embodiment. A configuration of the reception antenna 2 in the present embodiment is different from the first embodiment in the following point. In the present embodiment, the switching circuit 21 of the reception antenna 2 includes a first switching circuit unit 133 instead of the first switching circuit unit 33 in the first embodiment. As with the first switching circuit unit 33, the first switching circuit unit 133 is a switch circuit having the contacts 33a, 33b, 33c, and 33d. An arrangement of the first switching circuit unit 133 in the antenna unit 3 in terms of circuit configuration is the same as an arrangement of the first switching circuit unit 33 in the antenna unit 3 in terms of circuit configuration.

In the present embodiment, the cable 4 includes a third conductor 4c other than the first and second conductors 4a and 4b described in the first embodiment. One end of the third conductor 4c is connected to the second control line CL2. A third control line CL3 connecting the first switching circuit unit 133 and the other end of the third conductor 4c to each other is provided in the antenna unit 3.

In the present embodiment, the control unit 55 controls an opening and closing operation between the contacts 33a and 33c of the first switching circuit unit 133 and an opening and closing operation between the contacts 33b and 33d of the first switching circuit unit 133 by the second control line CL2, the third conductor 4c, and the third control line CL3.

As the cable 4 in the present embodiment, a two-core shielded cable or a three-core cable is used, for example. The reception antenna 2 in the present embodiment may include a first cable including the first and second conductors 4a and 4b and a second cable including the third conductor 4c in place of the cable 4.

### Modification

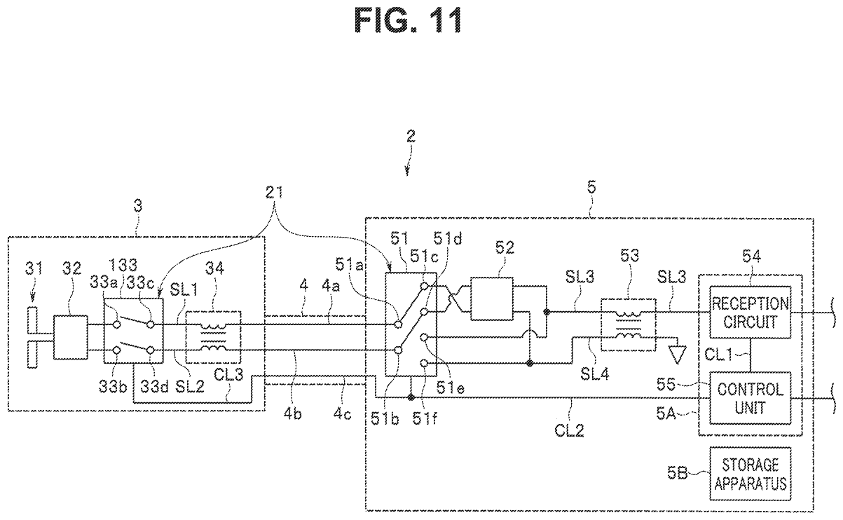

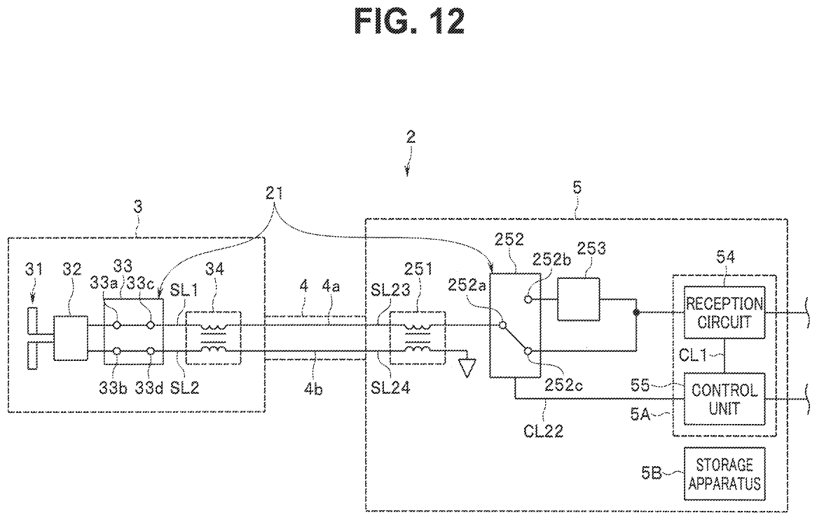

Next, a configuration of a modification of the reception antenna 2 is described. FIG. 11 is a functional block diagram illustrating the configuration of the modification of the reception antenna 2. In the reception antenna 2 illustrated in FIG. 10, a signal line connected to the contact 51c of the second switching circuit unit 51 is connected to the third signal line SL3 via the second connection circuit 52, and a signal line connected to the contact 51d of the second switching circuit unit 51 is connected to the fourth signal line SL4 via the second connection circuit 52. Meanwhile, in the modification, on the contrary to the reception antenna 2 illustrated in FIG. 10, the signal line connected to the contact 51c is connected to the fourth signal line SL4 via the second connection circuit 52, and the signal line connected to the contact 51d is connected to the third signal line SL3 via the second connection circuit 52.

Other configurations and effects in the present embodiment are similar to the configurations and effects in the first embodiment.

### Fourth Embodiment

Next, a fourth embodiment of the present invention is described with reference to FIG. 12. FIG. 12 is a functional block diagram illustrating a configuration of the reception antenna 2 in the present embodiment. The configuration of the reception antenna 2 in the present embodiment is different from the first embodiment in the following point. In the present embodiment, the switching circuit 21 of the reception antenna 2 includes a second switching circuit unit 252 instead of the second switching circuit unit 51 in the first embodiment. The reception unit 5 of the reception antenna 2 includes a second separation circuit 251, a second switching circuit unit 252, and a second connection circuit 253 instead of the second switching circuit unit 51, the second connection circuit 52, and the second separation circuit 53 in the first embodiment. The second separation circuit 251, the second switching circuit unit 252, and the second connection circuit 253 are arranged between the cable 4 and the reception circuit 54 in the stated order from the cable 4 side in terms of circuit configuration.

The second separation circuit 251 is a circuit for removing the noise signal transmitted to the cable 4 from the reception circuit 54. A signal line electrically connected to the first conductor 4a of the cable 4 in the reception unit 5 is referred to as a third signal line SL23, and a signal line electrically connected to the second conductor 4b of the cable 4 in the reception unit 5 is referred to as a fourth signal line SL24. For example, as illustrated in FIG. 12, the second separation circuit 53 may be a circuit in which a winding balun is inserted in the third and fourth signal lines SL23 and SL24. The winding balun may be a ferrite coil using ferrite for a core. Alternatively, the second separation circuit 251 may be a ferrite cable in which the third and fourth signal lines SL23 and SL24 are covered with ferrite. The fourth signal line SL24 is connected to the ground via the second separation circuit 251.

The second switching circuit unit 252 is a switch circuit having one contact 252a on the input side, and two contacts 252b and 252c on the output side. The second switching circuit unit 252 is configured to be able to select a connection destination of the contact 252a from the contacts 252b and 252c. FIG. 12 illustrates a state in which the contact 252a and the contact 252c are connected to each other.

In the present embodiment, a second control line CL22 connecting the second switching circuit unit 252 and the control unit 55 to each other is provided instead of the second control line CL2 in the first embodiment. The control unit 55 controls an operation of the second switching circuit unit 252 by the second control line CL22.

The third signal line SL23 is electrically connected to the contact 252a. In terms of circuit configuration, the second connection circuit 253 is positioned between the contact 252b of the second switching circuit unit 252 and the reception circuit 54. In other words, the contact 252b of the second switching circuit unit 252 is electrically connected to the reception circuit 54 via the second connection circuit 253. The contact 252c of the second switching circuit unit 252 is directly connected to the reception circuit 54.

As described in the first embodiment, the cable 4 can function as the reception means. The second connection circuit 253 is a circuit for connecting the cable 4 serving as the reception means to the electrical circuit of the reception antenna 2. The second connection circuit 253 may have a configuration similar to the configuration of the second connection circuit 52 illustrated in FIG. 5 in the first embodiment. In other words, the second connection circuit 253 may include a balun, a matching circuit, a filter, and an amplifier circuit. In terms of circuit configuration, the balun, the matching circuit, the filter, and the amplifier circuit are arranged in the stated order from the second switching circuit unit 252 side. The balun is electrically connected to the contact 252b of the second switching circuit unit 252. The amplifier circuit is electrically connected to the reception circuit 54. Functions of the balun, the matching circuit, the filter, and the amplifier circuit are the same as the functions of the balun 52A, the matching circuit 52B, the filter 52C, and the amplifier circuit 52D in the first embodiment.

Next, connection states realized by the switching circuit 21 in the present embodiment are described. As described in the first embodiment, the switching circuit 21 can perform switching between the first connection state, that is, the antenna element reception state in which the reception circuit 54, the antenna element 31, and the cable 4 are electrically connected to each other, and the second connection state, that is, the cable reception state in which the reception circuit 54 and the cable 4 are electrically connected to each other but the antenna element 31 is not electrically connected to the cable 4.

FIG. 12 illustrates the antenna element reception state. In the antenna element reception state, in the first switching circuit unit 33 of the switching circuit 21, the contact 33a and the contact 33c are connected to each other, and the contact 33b and the contact 33d are connected to each other. As a result, the antenna element 31 and the cable 4 are placed in a state of being electrically connected to each other via the first connection circuit 32, the first switching circuit unit 33, and the first separation circuit 34.

In the antenna element reception state, in the second switching circuit unit 252 of the switching circuit 21, the contact 252a and the contact 252c are connected to each other. As a result, the reception circuit 54 and the cable 4 are placed in a state of being electrically connected to each other via the second separation circuit 251 and the second switching circuit unit 252.

Although not shown, in the cable reception state, in the first switching circuit unit 33, a place between the contact 33a and the contact 33c is opened, and a place between the contact 33b and the contact 33d is opened. As a result, the antenna element 31 and the cable 4 are placed in a state of not being electrically connected to each other.

In the cable reception state, in the second switching circuit unit 252, the contact 252a and the contact 252b are connected to each other. As a result, the reception circuit 54 and the cable 4 are placed in a state of being electrically connected to each other via the second separation circuit 251, the second switching circuit unit 252, and the second connection circuit 253.

The reception circuit 54 and the cable 4 are electrically connected to each other without passing through the matching circuit of the second connection circuit 253 in the antenna element reception state, and are electrically connected to each other via the matching circuit in the cable reception state. Therefore, in the antenna element reception state, that is, when the cable 4 does not function as the reception means, the matching circuit does not perform impedance matching. In the cable reception state, that is, when the cable 4 functions as the reception means, the matching circuit performs impedance matching.

Description of the matching circuit above is also applicable to other components of the second connection circuit 253.

Other configurations and effects in the present embodiment are similar to the configurations and effects in the first embodiment.

The present invention is not limited to each of the abovementioned embodiments, and various changes, modifications, and the like can be made within the range in which the gist of the present invention is not changed. For example, the mobile body of the present invention is not limited to the capsule endoscope 101. More specifically, the mobile body of the present invention may be an implant medical device or a catheter.

What is claimed is:

1. A reception apparatus comprising:
a processor configured to perform predetermined processing on an electrical signal corresponding to a radio signal configured by a preamble portion and a data portion following the preamble portion, the data portion including image data picked up by a mobile body; and
a switching circuit configured to be capable of performing switching between a first connection state in which an antenna element capable of receiving the radio signal receives the radio signal, and a second connection state in which only a cable positioned between the processor and the antenna element and configured to transmit the electrical signal receives the radio signal transmitted by the mobile body, wherein the processor is configured to:
switch the switching circuit such that the first connection state is obtained in a first time period out of a time period in which the preamble portion is received and the second connection state is obtained in a second time period out of the time period in which the preamble portion is received, the second time period being different from the first time period; and
switch the switching circuit to either the first connection state or the second connection state based on the electrical signal corresponding to the radio signal received in each of the first time period and the second time period in a time period in which the data portion is received.

2. The reception apparatus according to claim 1, wherein the processor:
calculates an index relating to a reception performance of the radio signal based on the electrical signal corresponding to the radio signal received in each of the first time period and the second time period; and
switches the switching circuit to either the first connection state or the second connection state based on the index.

3. The reception apparatus according to claim 2, wherein:
the index is a reception strength of the radio signal; and
the processor switches the switching circuit to a connection state corresponding to a time period in which the radio signal including a larger reception strength is received out of the first time period and the second time period in the time period in which the data portion is received.

4. The reception apparatus according to claim 2, wherein:
the preamble portion includes identification data that changes with a predetermined pattern;
the index is a rate of concordance between reference data serving as a reference of the identification data and the identification data; and
the processor:
holds the reference data; and
switches the switching circuit to a connection state corresponding to a time period in which the radio signal including a higher rate of concordance is received out of the first time period and the second time period in the time period in which the data portion is received.

5. The reception apparatus according to claim 2, further comprising a plurality of reception antennas, wherein:
the plurality of reception antennas each include the antenna element, the cable, and the switching circuit; and
the processor selects a reception antenna that receives the data portion based on the index, and switches the switching circuit such that a selected connection state is obtained in the selected reception antenna in the time period in which the data portion is received.

6. The reception apparatus according to claim 1, wherein:
the radio signal includes a position detection portion for detecting a position of the mobile body; and
the processor switches the switching circuit such that the first connection state is obtained in a time period in which the position detection portion is received.

7. The reception apparatus according to claim 6, further comprising a plurality of reception antennas, wherein:
the plurality of reception antennas each include the antenna element, the cable, and the switching circuit; and
the processor switches the switching circuit such that the first connection state is obtained in all of the plurality of reception antennas in the time period in which the position detection portion is received.

8. The reception apparatus according to claim 1, wherein:
the switching circuit includes a first switching circuit positioned between the antenna element and the cable; and
the first switching circuit electrically connects the antenna element and the cable to each other in the first connection state and electrically disconnects the antenna element from an electrical circuit including the processor and the cable in the second connection state.

9. The reception apparatus according to claim 8, further comprising a matching circuit positioned between the processor and the cable, wherein:
the switching circuit further includes a second switching circuit positioned between the cable and the matching circuit; and
the second switching circuit electrically connects the processor and the cable to each other without passing through the matching circuit in the first connection state and electrically connects the processor and the cable via the matching circuit in the second connection state.

10. The reception apparatus according to claim 1, further comprising:
a first separation circuit for removing a noise signal transmitted to the antenna element from the cable, the first separation circuit being positioned between the antenna element and the cable; and
a second separation circuit for removing a noise signal transmitted to the cable from the processor, the second separation circuit being positioned between the processor and the cable.

11. The reception apparatus according to claim 1, wherein the mobile body is a capsule endoscope.

12. A reception method of receiving a radio signal configured by a preamble portion and a data portion following the preamble portion, the data portion including image data picked up by a mobile body, the radio signal being transmitted by the mobile body, the reception method comprising:
switching the switching circuit to a first connection state in which an antenna element capable of receiving the radio signal receives the radio signal in a first time period out of a time period in which the preamble portion is received;
switching the switching circuit to a second connection state in which only a cable positioned between a processor configured to perform predetermined processing on an electrical signal corresponding to the radio signal and the antenna element receives the radio signal in a second time period out of the time period in which the preamble portion is received, the second time period being different from the first time period, the cable being configured to transmit the electrical signal; and
switching the switching circuit to either the first connection state or the second connection state based on the electrical signal corresponding to the radio signal received in each of the first time period and the second time period in a time period in which the data portion is received.

13. A reception apparatus configured to receive a radio signal configured by a preamble portion and a data portion following the preamble portion, the data portion including image data picked up by a mobile body, the radio signal being transmitted by the mobile body, the reception apparatus comprising:
a processor configured to perform predetermined processing on an electrical signal corresponding to the radio signal;
a reception antenna configured by an antenna element capable of receiving the radio signal, and cable positioned between the processor and the antenna element and configured to transmit the electrical signal; and
a switching circuit capable of performing switching between a first connection state in which the antenna element receives the radio signal and a second connection state in which only the cable receives the radio signal, wherein the processor:
switches the switching circuit such that the first connection state is obtained in a first time period out of a time period in which the preamble portion is received and the second connection state is obtained in a second time period out of the time period in which the preamble portion is received, the second time period being different from the first time period; and
switches the switching circuit to either the first connection state or the second connection state based on the electrical signal corresponding to the radio signal received in each of the first time period and the second time period in a time period in which the data portion is received.
